(19) United States
(12) Patent Application Publication
(10) Pub. No.: US 2023/0192483 A1
(43) Pub. Date: Jun. 22, 2023

(54) PROCESS AND PLANT FOR PRODUCING METHANOL AND CARBON MONOXIDE

(71) Applicant: L'Air Liquide Société Anonyme pour l'Etude et l'Exploitation des Procédés Georges Claude, Paris (FR)

(72) Inventors: Peter WEIGAND, Bad Vilbel (DE); Chin Han LIM, Frankfurt am Main (DE); Veronika GRONEMANN, Karben (DE); Matthias STEIN, Dusseldorf (DE)

(21) Appl. No.: 18/080,365

(22) Filed: Dec. 13, 2022

(30) Foreign Application Priority Data
Dec. 20, 2021 (EP) 21020646.2

(51) Int. Cl. C01B 3/36; C01B 3/52; C01B 3/50; C01B 32/40; B01J 19/24; B01J 19/00; C07C 29/151; B01D 53/02; F25J 1/00

(52) U.S. Cl. CPC C01B 3/36; C01B 3/52; C01B 3/506; C01B 32/40; B01J 19/245; B01J 19/0013; C07C 29/1518; B01D 53/02; F25J 1/0027; C01B 2203/0255; C01B 2203/061; C01B 2203/0475; C01B 2203/047; C01B 2203/046; C01B 2203/0415; C01B 2203/0822; B01J 2219/00157; B01D 2256/20; B01D 2256/16; B01D 2257/504

(57) ABSTRACT

The present invention specifies a process and a plant for simultaneous production of methanol and pure carbon monoxide which includes synthesis gas production by partial oxidation of an input stream containing hydrocarbons and subsequent methanol synthesis. According to the invention carbon dioxide is separated from the raw synthesis gas using a sorption apparatus and at least partially introduced into the input gas for the methanol synthesis reactor.

### FIELD OF THE INVENTION

The present invention relates to a process for producing methanol and pure carbon monoxide from an input stream containing hydrocarbons. In particular the present invention relates to a process for simultaneous provision of synthesis gas for production of methanol and pure carbon monoxide using gaseous or liquid carbon-containing input material such as preferably natural gas but also heavy refinery residues and comparable carbon-containing residues in a partial oxidation process. The invention further relates to a plant for performing such a production process.

### BACKGROUND

Processes for industrial production of methanol by heterogeneously catalyzed conversion of synthesis gas or the hydrogen present therein in suitable synthesis reactors have long been known in the art. Synthesis gases are gas mixtures containing hydrogen and carbon oxides which are used in various synthesis reactions.

Methanol is an important indispensable feedstock chemical of the chemical industry for further processing into end products. Ullmann's Encyclopedia of Industrial Chemistry, Sixth Edition, 1998 Electronic Release, chapter “Methanol”, subchapter 5 “Process Technology” describes various basic processes for producing methanol.

A modern two-stage process for producing methanol is disclosed in European patent specification EP 0 790 226 B1 for example. The methanol is produced in a circular process wherein a mixture of fresh and partly reacted synthesis gas is supplied initially to a water-cooled reactor (WCR) and then to a gas-cooled reactor (GCR), in each of which the synthesis gas is converted over a copper-based fixed-bed catalyst to afford methanol. The methanol produced in the process is separated from the synthesis gas to be recycled which is then passed through the gas-cooled reactor in countercurrent as coolant and preheated to a temperature of 220° C. to 280° C. before it is introduced into the first synthesis reactor. A portion of the synthesis gas to be recycled is removed from the process as a purge stream to prevent inert components from accumulating in the synthesis circuit.

Unconverted methane from synthesis gas production is considered an inert component in the context of methanol synthesis since this compound does not undergo further conversion under the conditions of methanol or ammonia synthesis. The same applies to argon which passes into synthesis gas production via feed streams.

There are different processes for producing synthesis gas comprising hydrogen (H₂) and carbon oxides such as carbon monoxide (CO) and carbon dioxide (CO₂) as input gas for methanol synthesis, for example steam reforming, autothermal reforming (ATR), combinations thereof (so-called combined reforming) and noncatalytic partial oxidation (POX). Technical details of these processes are known in the art and are comprehensively described in, for example, Ullmann's Encyclopedia of Industrial Chemistry, Sixth Edition, 1998 Electronic Release, keyword “Gas Production”. In the further context and for the purposes of the present disclosure autothermal reforming is considered a partial oxidation process on account of the employed oxygen deficit relative to a total oxidation/complete combustion.

Starting materials for the abovementioned processes for synthesis gas production include hydrocarbons such as natural gas, comprising its main component methane or naphtha. The recited processes afford different ratios of the product components carbon monoxide (CO) and hydrogen (H₂) as is apparent from the following reaction equations:

2CH₄+O₂=2CO+4H₂ (partial oxidation)

2CH₄+½O₂+H₂O=2CO+5H₂ (autothermal reforming)

2CH₄+2H₂O=2CO+6H₂ (pure steam reforming)

Since partial oxidation or autothermal reforming is operated with an excess of hydrocarbon/deficiency of oxygen to inhibit the total oxidation of the hydrocarbons to carbon dioxide a synthesis gas is often obtained which has a hydrogen deficit having regard to its use as input gas for methanol synthesis. This necessitates according to the following reaction equation

2H₂+CO=CH₃OH

an H₂/CO ratio of at least 2 and under practical synthesis conditions often slightly greater than 2, for example 2.1. This ratio is typically formulated as the stoichiometry number SN of the methanol synthesis and takes into account that carbon dioxide too reacts to afford methanol.

SN=([H₂]−[CO₂])/([CO]+[CO₂])≥2 (e.g. 2.1)

By contrast, synthesis gases obtained by partial oxidation or autothermal reforming often have a stoichiometry number of ≤1.9, occasionally even ≤1.7 auf. Accordingly, none of the reforming/partial oxidation processes in themselves afford a synthesis gas product having the stoichiometric H₂/CO ratio of 2 or only a slight hydrogen excess desired for the methanol synthesis.

When the yield of hydrogen is to be maximized at the cost of the carbon monoxide it is customary to subject the raw synthesis gas to a CO conversion reaction which is also described as a water gas shift reaction (WGS) or CO shift reaction and proceeds according to the following reaction equation:

CO+H₂O=CO₂+H₂

The further workup of the produced raw synthesis gas usually also comprises a sorption process for separating further unwanted concomitants, for example by physical or chemical absorption or gas scrubbing. Such processes thus allow unwanted constituents, in particular carbon dioxide (CO₂), to be safely removed down to trace amounts from the desired main synthesis gas constituents hydrogen and carbon monoxide. A known and often employed process is the Rectisol process which comprises a scrubbing of the raw synthesis gas with cryogenic methanol as the absorbent and is likewise described in principle in the abovementioned document.

Cryogenic gas fractionation (so-called coldbox) may also be used to remove traces of higher hydrocarbons or of carbon monoxide. This employs mainly liquid methane or liquid nitrogen to absorb higher boiling gases such as carbon monoxide. Obtained offgas stream may be used as fuel gas or alternatively separated into a methane-rich gas stream and into a further carbon monoxide- and hydrogen-comprising gas stream by means of further cryogenic gas fractionation if desired or required. Further details of processes of cryogenic gas fractionation may be found in the literature; exemplary reference may be made for example to the textbook Haring, H. W., Industrial Gases Processing, WILEY-VCH Verlag, Weinheim (2008), Chapter 5.2.3.6 “Cryogenic Separation Processes”.

Production processes for simultaneous production of methanol and carbon monoxide are already well known from the prior art. U.S. Pat. No. 6,232,352 B1 describes a process for simultaneous production of CO and methanol for production of acetic acid by steam reforming. In this process the firing of the steam reformer and the fired heater for steam production have the result that a large amount of carbon dioxide is emitted to the atmosphere. The CO₂ emission may be more than 5 kg of CO₂ per kg of methanol product.

Published patent DE 10214003 B4 describes a process for coproduction of carbon monoxide and methanol with low, if any, CO₂ emission through catalytic or noncatalytic partial oxidation of a gaseous or liquid input material using oxygen and hydrogen, wherein a portion of the produced synthesis gas is diverted and the carbon dioxide present in this gas is separated via a gas scrubbing, recompressed and recycled into the synthesis gas reactor via the feed injector or a similar apparatus.

In the prior art processes for producing synthesis gas for methanol and for producing CO by steam reforming the heat for the reforming process derives from the combustion of fossil fuels, generally natural gas, where a considerable amount of CO₂ is liberated. The separation of this CO₂ by separation and storage, for example underground storage (Carbon Capture and Storage, CCS) is possible in principle but requires considerable technical complexity and energy expenditure as well as a destination for storage and a means of transport thereto.

In the above-described patent specification DE 10214003 B4 for coproduction of CO and methanol the CO₂ generated is at least partially recycled to the partial oxidation stage, thus resulting in low CO₂ emission. However, this process requires at least the compression of two CO₂-containing process streams. Furthermore, a portion of the CO₂ supplied to the partial oxidation reactor is converted into CO which is disadvantageous in many cases since it is known that efficient conversion in the methanol synthesis requires a stoichiometry number of about 2, wherein values between 2.0 and 2.2 are regarded as optimal. CO reduces the stoichiometry number according to the calculation formula specified above. Especially when using a noncatalytic partial oxidation (POX), the CO₂ content in the synthesis gas is generally excessively low for an optimal input gas for methanol synthesis. Generally, a noncatalytic process achieves lower CO₂ contents of the synthesis gas (typically 2% to 4% by volume) than the use of a catalytic process (typically 6% to 8% by volume, where % by volume values are in each case on a dry basis at the outlet of the reactor). This synthesis gas is generally diluted with recycle gas from the synthesis gas circuit of the methanol synthesis at the reactor inlet of the methanol synthesis reactor.

In the case of the POX process with natural gas as input gas described in the abovementioned patent publication the CO₂ content at the reactor inlet of the methanol synthesis reactor resulting from the partial oxidation would therefore be excessively low which would result in a markedly lower efficiency of the methanol synthesis.

### SUMMARY

It is accordingly an object of the present invention to specify a process and a plant which does not exhibit the described disadvantages of the prior art and which especially makes it possible in a process for simultaneous production of methanol and pure carbon monoxide to achieve material and/or energy utilization of ideally all material streams generated. The invention shall moreover make it possible to achieve an optimal adjustment of the stoichiometry number for the methanol synthesis without import of hydrogen not produced in the process.

This object is achieved in a first aspect of the invention by a process having the features of claim 1 and by a plant having the features of claim 12. Further embodiments according to further aspects of the invention are apparent from the subsidiary claims of the respective category.

Partial oxidation conditions or methanol synthesis conditions are to be understood as meaning the process conditions known per se to a person skilled in the art, in particular of temperature, pressure and residence time, as mentioned for example hereinabove and discussed in detail in the relevant literature, for example patent specification DE 10214003 B4, and under which at least partial conversion, but preferably industrially relevant conversions of the reactants into the products of the respective process, takes place. The same applies to the selection of a suitable catalyst in the case of methanol synthesis/autothermal reforming. Corresponding partial oxidation reactors/methanol synthesis reactors are known per se to those skilled in the art and described for example in the literature described at the outset.

A sorption apparatus in the context of the present disclosure is to be understood as meaning an apparatus which makes it possible for a fluid mixture, for example a gas mixture, to be separated into its constituents or for unwanted components to be separated from the mixture by means of a physical or chemical sorption process using a suitable sorbent. The sorption process may be based on an adsorption, i.e. a bonding of the substance(s) to be separated onto a surface or interface of the solid absorbent, or on an absorption, i.e. a taking-up of the substance(s) to be separated into the volume of the liquid or solid absorbent. The substance(s) removed and bound by sorption are referred to as adsorbate/absorbate. The binding forces acting here may be physical or chemical by nature. Accordingly, physical sorption results from usually relatively weak, less specific bonding forces, for example van der Waals forces, whereas chemical sorption results from relatively strong, more specific bonding forces, and the adsorbate/absorbate and/or the adsorbent/absorbent is/are chemically altered.

One specific, physical absorption process is gas scrubbing with cryogenic methanol, which uses as absorbent or scrubbing medium methanol having a temperature cooled by means of refrigerating processes to below ambient temperature, preferably below 0° C., most preferably below −30° C. This process is known to those skilled in the art as the Rectisol process.

In connection with the present invention dividing a material stream is to be understood as meaning splitting of the stream into at least two substreams whose composition of matter and phase state correspond to that of the starting stream. By contrast, separating a material stream is to be understood as meaning splitting of the stream into at least two substreams with the aid of a phase equilibrium, wherein the compositions of the obtained material streams differ from one another and from that of the starting stream.

For the purposes of this description, steam is to be understood as being synonymous with water vapour unless stated otherwise in an individual case. By contrast, the term “water” refers to water in the liquid state of matter unless otherwise stated in an individual case.

A means is understood to mean an article which makes it possible to achieve, or is helpful in achieving, an objective. In particular, means of performing a particular process step are understood to mean all those physical articles which a person skilled in the art would consider in order to be able to perform this process step. For example, a person skilled in the art will consider means of introducing or discharging a material stream to include all transporting and conveying apparatuses, i.e. for example pipelines, pumps, compressors, valves and the corresponding openings in container walls which seem necessary or sensible to said skilled person for performance of this process step on the basis of his knowledge of the art.

Fluid connection between two regions or plant components is to be understood here as meaning any kind of connection that enables flow of a fluid, for example a reaction product or a hydrocarbon fraction, from one to the other of the two regions, irrespective of any interposed regions, components or required conveying means.

All approximate pressures are reported in bar as absolute pressure units, bara for short, or in gauge pressure units, barg for short, unless otherwise stated in the particular individual context.

The invention is based on the finding that starting from the process taught in patent specification DE 10214003 B4, and while maintaining the marked reduction in CO₂ emissions, improved utilization of the separated CO₂ and further advantages are obtainable. This is achieved according to the invention when the carbon dioxide separated from the raw synthesis gas substream using the sorption apparatus is at least partially introduced into the input gas for the methanol synthesis reactor instead of being recycled to the partial oxidation stage. This results in the following advantages:

(a) The CO₂ content in the input gas for the methanol synthesis is increased and the stoichiometry number SN can thus be adjusted to the value of slightly more than 2, for example 2.1, desired for methanol synthesis. This results in improved conversion in the methanol synthesis reactor and a higher efficiency of methanol production, in particular in processes with synthesis gas production by partial oxidation, for example using natural gas POX where the CO₂ content in the syngas is, per se, relatively low.

(b) The technical complexity and energy demand for the CO₂ compression and the cooling of the compressor are reduced. Depending on the CO/methanol ratio the energy demand falls by about 15% to 20%.

(c) The oxygen demand is reduced.

(d) Controlling the introduction of the separated CO₂ into the input gas for the methanol synthesis is less complex than the recycling and introduction thereof into the partial oxidation reactor.

By introducing the separated CO₂ directly into the input gas for the methanol synthesis the CO₂ content at the inlet into the methanol synthesis reactor is increased. Especially when lighter hydrocarbons such as natural gas are used as starting material in a partial oxidation process this can make it possible to achieve a more advantageous CO₂ content in the input gas for the methanol synthesis. The energy for the post-compression and the cooling of the compressor is lower than in the process taught in patent specification DE 10214003 B4 since the CO₂ molar flow and the pressure at the inlet into the methanol synthesis reactor are lower than in the case of the partial oxidation reactor. Since the CO₂ need not be heated to the partial oxidation temperature, less oxygen is also required. This is also the case when slightly more natural gas is used to counter the slightly lower raw synthesis gas stream from the partial oxidation reactor when no CO₂ and therefore less carbon is passed into the partial oxidation reactor.

In a second aspect of the invention the process according to the invention is characterized in that the hydrogen-rich gas stream is at least partially introduced into the methanol synthesis reactor. This provides a further opportunity for adjusting the desired stoichiometry number SN for the methanol synthesis.

In a third aspect of the invention the process according to the invention is characterized in that a proportion of the hydrogen-rich gas stream such that the stoichiometry number SN which relates to the entirety of all material streams introduced into the methanol synthesis reactor at the reactor inlet of the methanol synthesis reactor is between 1.8 and 2.4, preferably between 2.0 and 2.2, is introduced into the methanol synthesis reactor. This provides a further opportunity for adjusting the optimal stoichiometry number SN for the methanol synthesis.

In a fourth aspect of the invention the process according to the invention is characterized in that the hydrogen-rich gas stream is at least partially supplied to the burner of the heating apparatus as a second heating gas. This makes it possible to reduce the CO₂ emission of the overall process since carbon-based fuel is partially substituted with hydrogen.

In a fifth aspect of the invention the process according to the invention is characterized in that a portion of the input stream containing hydrocarbons, preferably natural gas, is supplied to the burner of the heating apparatus as a third heating gas. This makes it possible to reliably provide heating gas during startup of the process/the plant since flammable waste streams such as for example the methanol synthesis purge stream are only available after startup of the overall process, in particular of the methanol synthesis.

In a sixth aspect of the invention the process according to the invention is characterized in that at least a portion of the methanol synthesis purge stream is supplied to the burner of the heating apparatus as a fourth heating gas. This allows thermal utilization of the methanol synthesis purge stream and hazardous substances present therein, for example carbon monoxide, are neutralized. Flexibility in terms of the available heating gases is also increased.

In a seventh aspect of the invention the process according to the invention is characterized in that the heating apparatus is used for steam production, wherein the steam produced is at least partially used as a moderator in the partial oxidation stage. This allows the waste heat to be better utilized and the thermal efficiency of the process/the plant is increased.

In an eighth aspect of the invention the process according to the invention is characterized in that the heating apparatus is used for steam production, wherein the steam produced is at least partially provided to external consumers (export steam). This reduces the technical complexity and energy expenditure for steam production for the external consumers.

In a ninth aspect of the invention the process according to the invention is characterized in that a carbon dioxide-containing gas stream deriving from a process-external source is additionally introduced into the methanol synthesis reactor. This provides a sink for the climate-damaging carbon dioxide.

In a tenth aspect of the invention the process according to the invention is characterized in that the heating apparatus is used for preheating the input stream containing hydrocarbons and/or the stream of the oxygen-containing oxidant. This allows the waste heat to be better utilized and the thermal efficiency of the process/the plant is increased.

In an eleventh aspect of the invention the process according to the invention is characterized in that the methanol synthesis purge stream is separated using a separation apparatus, preferably a membrane separation apparatus, into a first purge stream enriched in hydrogen and into a second purge stream depleted in hydrogen and enriched in carbon oxides and methane, wherein at least a portion of the first purge stream enriched in hydrogen is supplied to the burner of the heating apparatus as a fourth heating gas and wherein at least a portion of the second purge stream enriched in carbon oxides and methane is passed to the partial oxidation stage. This allows thermal and material utilization of the methanol synthesis purge stream and the CO₂ emission of the process/the plant is reduced.

In a thirteenth aspect of the invention the plant according to the invention is characterized in that it further comprises means which allow the hydrogen-rich gas stream to be at least partially supplied to the burner of the heating apparatus as a second heating gas.

The technical effect and advantages associated with this aspect correspond to those discussed in connection with the fourth aspect of the invention.

In a fourteenth aspect of the invention the plant according to the invention is characterized in that it further comprises means which allow a portion of the input stream containing hydrocarbons, preferably natural gas, to be supplied to the burner of the heating apparatus as a third heating gas.

The technical effect and advantages associated with this aspect correspond to those discussed in connection with the fifth aspect of the invention.

In a fifteenth aspect of the invention the plant according to the invention is characterized in that it further comprises means which allow at least a portion of the methanol synthesis purge stream to be supplied to the burner of the heating apparatus as a fourth heating gas.

The technical effect and advantages associated with this aspect correspond to those discussed in connection with the sixth aspect of the invention.

In a sixteenth aspect of the invention the plant according to the invention is characterized in that it further comprises means which allow the heating apparatus to be used for steam production, wherein the steam produced is at least partially usable as a moderator in the partial oxidation stage.

The technical effect and advantages associated with this aspect correspond to those discussed in connection with the seventh aspect of the invention.

In a seventeenth aspect of the invention the plant according to the invention is characterized in that it further comprises means which allow the heating apparatus to be used for steam production, wherein the steam produced is at least partially providable to external consumers (export steam).

The technical effect and advantages associated with this aspect correspond to those discussed in connection with the eighth aspect of the invention.

In an eighteenth aspect of the invention the plant according to the invention is characterized in that it further comprises means which allow a carbon dioxide-containing gas stream deriving from a process-external source to be additionally introducible into the methanol synthesis reactor.

The technical effect and advantages associated with this aspect correspond to those discussed in connection with the ninth aspect of the invention.

In a nineteenth aspect of the invention the plant according to the invention is characterized in that it further comprises means which allow the heating apparatus to be usable for preheating the input stream containing hydrocarbons and/or the stream of the oxygen-containing oxidant.

The technical effect and advantages associated with this aspect correspond to those discussed in connection with the tenth aspect of the invention.

In a twentieth aspect of the invention the plant according to the invention is characterized in that it further comprises means which allow
the methanol synthesis purge stream to be separable using a separation apparatus, preferably a membrane separation apparatus, into a first purge stream enriched in hydrogen and into a second purge stream depleted in hydrogen and enriched in carbon oxides and methane,
at least a portion of the first purge stream enriched in hydrogen to be suppliable to the burner of the heating apparatus as a fourth heating gas,
at least a portion of the second purge stream enriched in carbon oxides and methane to be suppliable to the partial oxidation stage.

The technical effect and advantages associated with this aspect correspond to those discussed in connection with the eleventh aspect of the invention.

### BRIEF DESCRIPTION OF THE DRAWING

Developments, advantages and possible applications of the invention are also apparent from the following description of working and numerical examples and the drawings. The invention is formed by all of the features described and/or depicted, either on their own or in any combination, irrespective of the way they are combined in the claims or the dependency references therein.

FIG. 1 a schematic representation of the process/the plant according to one embodiment of the invention.

### DETAILED DESCRIPTION OF PREFERRED EMBODIMENTS

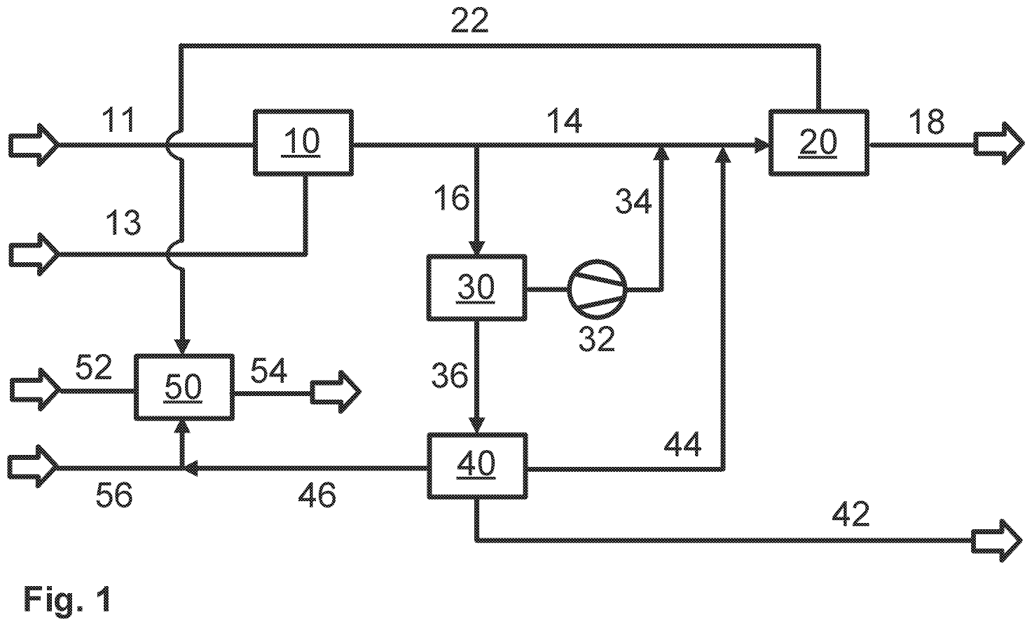

In the configuration of a process/a plant according to the invention shown in FIG. 1 conduit 11 supplies an input stream containing hydrocarbons, for example natural gas, in a preferred example natural gas having a methane content of at least 80% by volume, to a noncatalytic partial oxidation stage (POX) 10. The oxygen required as an oxidant for the partial oxidation is supplied to the partial oxidation stage via conduit 13. The noncatalytic partial oxidation stage is operated under partial oxidation conditions known per se. As an additional operating medium the POX stage is optionally supplied with steam and/or carbon dioxide as moderator.

In a further example (not shown) the partial oxidation stage may be in the form of an autothermal reformer (ATR) which in one example is operated at a pressure of 60 bara. As an additional operating medium the ATR is optionally supplied with steam and/carbon monoxide as moderator.

The partial oxidation stage 10 carries out an at least partial conversion of the input stream containing hydrocarbons under synthesis gas production conditions to afford a raw synthesis gas stream containing hydrogen (H₂), carbon monoxide (CO) and components inert in the context of methanol synthesis such as methane (CH₄) which is discharged from the partial oxidation stage and divided into a first raw synthesis gas substream (conduit 14) and into a second raw synthesis gas substream (conduit 16).

Via conduit 14 the first raw synthesis gas substream is supplied to a methanol synthesis reactor 20, in which there follows an at least partial conversion of the first raw synthesis gas substream under methanol synthesis conditions. The resulting raw methanol product is via conduit 18 discharged from the methanol synthesis reactor 20 and sent for further processing, workup, storage or to a consumer.

For the purposes of the present description the term “methanol synthesis reactor” and the reference numeral 20 are to be understood as meaning that they comprise not only the catalytic reactor(s) for methanol synthesis but also further customary constituents of a methanol synthesis unit familiar to those skilled in the art (not shown):
conduits and at least one compressor for construction of a circuit for unconverted synthesis gas,
coolers for cooling the reactor product stream of the methanol synthesis reactor,
a phase separation apparatus for separating the cooled reactor product stream of the methanol synthesis reactor into a first liquid product stream and a first residual gas stream containing unconverted synthesis gas constituents and inert components,
an apparatus for dividing the first residual gas stream into a methanol synthesis purge stream and into a recycle stream which is recycled to the methanol synthesis reactor.

The methanol synthesis purge stream is discharged from the methanol synthesis reactor via conduit 22.

The second raw synthesis gas substream is introduced into a sorption apparatus 30 for removal of carbon dioxide via conduit 16. In one example the sorption apparatus operates according to a physical sorption process and cryogenic methanol is used as the absorbent/scrubbing medium (Rectisol process). Details of this process are known to those skilled in the art. This results in further synergistic advantages since in one example a portion of the methanol produced in the methanol synthesis reactor 20 may be used as scrubbing medium. In one example portions of the apparatuses for workup of the raw methanol product discharged from the methanol synthesis reactor 20 via conduit 18 may also be used for regenerating the scrubbing medium laden with carbon dioxide. In one example waste heat from the apparatuses for workup of the raw methanol product may be used for heating or preheating of the scrubbing medium laden with carbon dioxide, for example for the purposes of regeneration.

For the purposes of the present description the term “sorption apparatus” and the reference numeral 30 are to be understood as meaning that they comprise not only the actual removal/separation of carbon dioxide but also the regeneration of the employed sorption medium and the production of a carbon dioxide-enriched gas stream. The carbon dioxide-enriched gas stream is compressed to methanol synthesis pressure in a compressor 32 arranged in the conduit path of the conduit 34 and according to the invention supplied to the methanol synthesis reactor 20 via conduit 34. In one example conduit 34 opens into conduit 14, by means of which the first raw synthesis gas substream is introduced into the methanol synthesis reactor. In one example conduit 34 opens directly into the methanol synthesis reactor.

A carbon dioxide-depleted synthesis gas stream is discharged from the sorption apparatus 30 via conduit 36, passed to a cryogenic gas fractionation stage 40 and introduced thereto. The cryogenic gas fractionation stage separates the carbon dioxide-depleted synthesis gas stream into the following substreams:

(1) A carbon monoxide-rich gas stream. This stream is discharged from the process as a carbon monoxide product stream via conduit 42.

(2) A hydrogen-rich gas stream. This is supplied via conduit 44 to the methanol synthesis reactor 20 and used therein to establish the desired stoichiometry number for the methanol synthesis. In one example the stoichiometry number thus established is between 1.8 and 2.4, preferably between 2.0 and 2.2. In one example the stoichiometry number thus established is 2.1.

In one example (not shown) the hydrogen-rich gas stream is at least partially supplied to a burner of a heating apparatus 50 as heating gas. In one example (not shown) the entire hydrogen-rich gas stream is supplied to the burner of the heating apparatus 50 as heating gas and in one example (not shown) the proportion of the hydrogen-rich gas stream not required for establishing the desired stoichiometry number for the methanol synthesis is supplied to the burner of the heating apparatus 50 as heating gas.

In one example conduit 44 opens into conduit 14, by means of which the first raw synthesis gas substream is introduced into the methanol synthesis reactor. In one example conduit 44 opens directly into the methanol synthesis reactor.

(3) An offgas stream containing inert components, methane, hydrogen and carbon monoxide. This stream is at least partially supplied as heating gas to a burner of a heating apparatus 50 via conduit 46. The burner of the heating apparatus 50 is further supplied via conduit 56 with an input stream containing hydrocarbons, for example natural gas, as fuel gas. In one example the burner of the heating apparatus 50 is further supplied via conduit 22 with at least a portion of the methanol synthesis purge stream from the methanol synthesis reactor as fuel gas.

In one example the heating apparatus 50 is used for steam production. To this end boiler feed water is introduced into the heating apparatus 50 via conduit 52 and evaporated therein. The steam produced is discharged from the heating apparatus 50 via conduit 54. In one example a portion of the steam produced is used as moderator in the partial oxidation stage 10. In one example at least a portion of the steam produced is provided to external consumers (export steam).

Further advantages and an even more flexible process mode result from the following examples which are combinable with the basic process according to the invention:

In one example carbon dioxide from a process-external CO₂ source is additionally introduced into the partial oxidation stage. This is especially advantageous when the process-external CO₂ stream is available at elevated pressure so that compression before introduction into the partial oxidation stage is minimized or even completely avoided.

In one example carbon dioxide from a process-external CO₂ source is additionally introduced into the methanol synthesis reactor.

In one example hydrogen from a process-external hydrogen source is used to adjust the desired stoichiometry number for the methanol synthesis.

### Numerical Example

The following table shows a comparison of calculated parameters of the invention with a process scheme according to the prior art (DE 10214003 B34) for a predetermined production amount of CO and methanol.

TABLE
Comparison of calculated parameters of the invention with a
process scheme according to the prior art (DE 10214003 B4)
for a predetermined production amount of CO and methanol.

| | | Comparative | Invention |
|---|---|---|---|
| CO production | kg/h | 14560 | 14558 |
| MeOH production | kg/h | 34296 | 34308 |
| Natural gas feed | kg/h | 27914 | 27999 |
| Oxygen feed | kg/h | 35860 | 35621 |
| Kg of syngas/kg of | | 2.071 | 2.015 |
| Synthesis gas to | kg/h | 25949 | 2582 |
| Synthesis gas to | kg/h | 31861 | 30585 |
| CO2 recycling | kg/h | 2181 | 1938 |
| Compressor power | kW | 160 | 130 |
| Compressor cooling | kW | 140 | 117 |
| MeOH feed stream | kg/h | 36856 | 3764 |
| Stoichiometry | | 2.085 | 2.086 |
| CO2 to MeOH | mol % | 1.76% | 2.74% |
| CO to MeOH | mol % | 29.74% | 28.43% |
| H2 to MeOH | mol % | 67.43% | 67.75% |
| CO/CO2 to MeOH | | 16.9 | 10.4 |
| Superheated steam | kg/h | 127431 | 124781 |

The invention achieves the following advantages over the prior art:
provision of a gas having a higher CO₂ content which is more suitable for methanol synthesis results in a higher efficiency of the methanol synthesis.
Less CO₂ requires compression to a lower pressure. The saving in terms of compressor power and coaling power is about 15% to 20%.
The oxygen demand falls by about 0.5% to 0.8%.
An excess steam production which is about 2% lower may be advantageous when no external utilization of steam and thus no steam export is desired.

### LIST OF REFERENCE SYMBOLS

[10] Partial oxidation stage
[11] Conduit
[13] Conduit
[14] Conduit
[16] Conduit
[18] Conduit
[20] Methanol synthesis reactor
[22] Conduit
[30] Sorption apparatus
[32] Compressor
[34] Conduit
[36] Conduit
[40] Cryogenic gas fractionation stage
[42] Conduit
[44] Conduit
[46] Conduit
[50] Heating apparatus
[52] Conduit
[54] Conduit
[56] Conduit

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

1. A process for producing methanol and pure carbon monoxide from an input stream containing hydrocarbons, comprising:
(a) providing an input stream containing hydrocarbons,
(b) supplying the input stream containing hydrocarbons to a partial oxidation stage,
(c) at least partially reacting the input stream containing hydrocarbons in the partial oxidation stage with a stream of an oxygen-containing oxidant under partial oxidation to afford a raw synthesis gas stream containing hydrogen, carbon monoxide, carbon dioxide and methane,
(d) discharging the raw synthesis gas stream from the synthesis gas production plant and dividing the raw synthesis gas stream into a first raw synthesis gas substream and into a second raw synthesis gas substream,
(e) introducing at least a portion of the first raw synthesis gas substream into a methanol synthesis reactor, at least partially converting the first raw synthesis gas substream in the methanol synthesis reactor under methanol synthesis conditions,
(f) discharging a methanol-containing first reactor product stream from the methanol synthesis reactor, cooling the first reactor product stream to below its dew point and separating the cooled first reactor product stream in a phase separation apparatus into a first liquid product stream and a first residual gas stream containing unconverted synthesis gas constituents and inert components, discharging the first liquid product stream from the process as a raw methanol product stream,
(g) dividing the first residual gas stream into a methanol synthesis purge stream and into a recycle stream which is recycled to the methanol synthesis reactor,
(h) introducing at least a portion of the second raw synthesis gas substream into a sorption apparatus for removal of carbon dioxide using a physical or chemical sorption process, discharging a carbon dioxide-depleted synthesis gas stream and a carbon dioxide-enriched gas stream from the sorption apparatus,
(j) introducing at least a portion of the carbon dioxide-depleted synthesis gas stream into a cryogenic gas fractionation stage, separating the carbon dioxide-depleted synthesis gas stream in the cryogenic gas fractionation stage into the following substreams:
(j1) a carbon monoxide-rich gas stream which is discharged from the process as a carbon monoxide product stream,
(j2) a hydrogen-rich gas stream,
(j3) an offgas stream containing methane, hydrogen and carbon monoxide which is at least partially supplied to a burner of a heating apparatus as a first heating gas,
wherein the carbon dioxide-enriched gas stream is at least partially introduced into the methanol synthesis reactor.

2. The process according to claim 1, wherein the hydrogen-rich gas stream is at least partially introduced into the methanol synthesis reactor.

3. The process according to claim 2, wherein a proportion of the hydrogen-rich gas stream such that the stoichiometry number which relates to the entirety of all material streams introduced into the methanol synthesis reactor at the reactor inlet of the methanol synthesis reactor is between 1.8 and 2.4 is introduced into the methanol synthesis reactor.

4. The process according to claim 1, wherein the hydrogen-rich gas stream is at least partially supplied to the burner of the heating apparatus as a second heating gas.

5. The process according to claim 1, wherein a portion of the input stream containing hydrocarbons is supplied to the burner of the heating apparatus as a third heating gas.

6. The process according to claim 1, wherein at least a portion of the methanol synthesis purge stream is supplied to the burner of the heating apparatus as a fourth heating gas.

7. The process according to claim 1, wherein the heating apparatus is used for steam production, wherein the steam produced is at least partially used as a moderator in the partial oxidation stage.

8. The process according to claim 1, wherein the heating apparatus is used for steam production, wherein the steam produced is at least partially provided to external consumers.

9. The process according to claim 1, wherein a carbon dioxide-containing gas stream deriving from a process-external source is additionally introduced into the methanol synthesis reactor.

10. The process according to claim 1, wherein the heating apparatus is used for preheating the input stream containing hydrocarbons and/or the stream of the oxygen-containing oxidant.

11. The process according to claim 1, wherein the methanol synthesis purge stream is separated using a separation apparatus into a first purge stream enriched in hydrogen and into a second purge stream depleted in hydrogen and enriched in carbon oxides and methane, wherein at least a portion of the first purge stream enriched in hydrogen is supplied to the burner of the heating apparatus as a fourth heating gas and wherein at least a portion of the second purge stream enriched in carbon oxides and methane is passed to the partial oxidation stage.

12. A plant for producing methanol and pure carbon monoxide from an input stream containing hydrocarbons, comprising the following constituents in fluid connection with one another:
(a) a means for providing the input stream containing hydrocarbons,
(b) a means for supplying the input stream containing hydrocarbons to a partial oxidation stage,
(c) a partial oxidation stage, a means for supplying a stream of an oxygen-containing oxidant to the partial oxidation stage, a means for discharging a raw synthesis gas stream containing hydrogen, carbon monoxide, carbon dioxide and methane from the partial oxidation stage,
(d) a means for dividing the raw synthesis gas stream into a first raw synthesis gas substream and into a second raw synthesis gas substream,
(e) a methanol synthesis reactor, a means for introducing at least a portion of the first raw synthesis gas substream into the methanol synthesis reactor,
(f) a means for discharging a methanol-containing first reactor product stream from the methanol synthesis reactor, a means for cooling the first reactor product stream to below its dew point, a phase separation apparatus for separating the cooled first reactor product stream into a first liquid product stream and a first residual gas stream containing unconverted synthesis gas constituents and inert components, a means for discharging the first liquid product stream from the process as a raw methanol product stream,
(g) a means for dividing the first residual gas stream into a methanol synthesis purge stream and into a recycle stream which is recycled to the methanol synthesis reactor,
(h) a sorption apparatus for removal of carbon dioxide using a physical or chemical sorption process, a heating apparatus having at least one burner, a means for introducing at least a portion of the second raw synthesis gas substream into the sorption apparatus, a means for discharging a carbon dioxide-depleted synthesis gas stream and a carbon dioxide-enriched gas stream from the sorption apparatus,
(j) a cryogenic gas fractionation stage suitable for separation of the carbon dioxide-depleted synthesis gas stream in the cryogenic gas fractionation stage into the following substreams:
(j1) a carbon monoxide-rich gas stream which is dischargeable from the process as a carbon monoxide product stream,
(j2) a hydrogen-rich gas stream,
(j3) an offgas stream containing methane, hydrogen and carbon monoxide which is at least partially introduceable to the at least one burner of the heating apparatus as a first heating gas, means for introducing at least a portion of the carbon dioxide-depleted synthesis gas stream into the cryogenic gas decomposition stage,
further comprising a means which allow the carbon dioxide-enriched gas stream to be at least partially introduced into the methanol synthesis reactor.

13. The plant according to claim 12, further comprising a means which allow the hydrogen-rich gas stream to be at least partially supplied to the burner of the heating apparatus as a second heating gas.

14. The plant according to claim 12, further comprising a means which allow a portion of the input stream containing hydrocarbons to be supplied to the burner of the heating apparatus as a third heating gas.

15. The plant according to claim 12, further comprising a means which allow at least a portion of the methanol synthesis purge stream to be supplied to the burner of the heating apparatus as a fourth heating gas.

16. The plant according to claim 12, further comprising a means which allow the heating apparatus to be used for steam production, wherein the steam produced is at least partially usable as a moderator in the partial oxidation stage.

17. The plant according to claim 12, further comprising a means which allow the heating apparatus to be used for steam production, wherein the steam produced is at least partially providable to external consumers.

18. The plant according to claim 12, further comprising a means which allow a carbon dioxide-containing gas stream deriving from a process-external source to be additionally introducible into the methanol synthesis reactor.

19. The plant according to claim 12, further comprising a means which allow the heating apparatus to be usable for preheating the input stream containing hydrocarbons and/or the stream of the oxygen-containing oxidant.

20. A plant according claim 12, further comprising a means which allow
the methanol synthesis purge stream to be separable using a separation apparatus, preferably a membrane separation apparatus, into a first purge stream enriched in hydrogen and into a second purge stream depleted in hydrogen and enriched in carbon oxides and methane,
at least a portion of the first purge stream enriched in hydrogen to be suppliable to the burner of the heating apparatus as a fourth heating gas,
at least a portion of the second purge stream enriched in carbon oxides and methane to be suppliable to the partial oxidation stage.
